(19) United States
(12) Patent Application Publication
(10) Pub. No.: US 2026/0250760 A1
(43) Pub. Date: Aug. 27, 2026

(54) BACTERIAL DNA DEPLETION TECHNIQUE TO IMPROVE THE EFFICIENCY OF NGS SEQUENCING OF EUKARYOTIC DNA

(71) Applicant: Color Health, Inc., Burlingame, CA (US)

(72) Inventor: Scott Topper, Burlingame, CA (US)

(73) Assignee: Color Health, Inc., Burlingame, CA (US)

(21) Appl. No.: 19/546,024

(22) Filed: Feb. 20, 2026

Related U.S. Application Data
(60) Provisional application No. 63/763,165, filed on Feb. 25, 2025.

(51) Int. Cl. C12Q 1/6874; C12Q 1/6806

(52) U.S. Cl. CPC C12Q 1/6874; C12Q 1/6806

(57) ABSTRACT

Some embodiments relate to techniques for enriching eukaryotic DNA in a DNA sample containing eukaryotic DNA and prokaryotic DNA. A DNA sample comprising eukaryotic DNA and prokaryotic DNA can be accessed. The DNA sample can be treated with one or more restriction enzymes, where at least one of the one or more restriction enzymes is a methylation-specific restriction enzyme that selectively digests prokaryotic DNA by targeting methylation patterns characteristic of prokaryotic genomes, the methylation patterns being absent or rare in eukaryotic DNA. The treated DNA sample can be sequenced.

### CROSS-REFERENCE TO RELATED APPLICATIONS

This application claims the benefit of and the priority to U.S. Provisional Patent Application No. 63/763,165, filed on Feb. 25, 2025, which is hereby incorporated by reference for all purposes.

### BACKGROUND

In research and clinical genetic analysis, cross-species contamination of DNA samples during next-generation sequencing (NGS) imposes significant financial and technical burdens, particularly when it compromises the accuracy of results. Contaminated samples have higher failure rates, require additional sequencing to provide sufficient analytic power for the species of interest, and can even yield misleading data, all of which can necessitate costly repeat experiments or additional rounds of sequencing to achieve reliable results. In clinical diagnostics and research, where precision is critical, the presence of foreign DNA can obscure the true genetic signals of interest, leading to wasted time and resources. For large-scale projects, contamination can result in substantial financial losses as labs struggle to rectify the problem or compensate for corrupted datasets.

The accuracy of sequencing data can be impacted by contamination. NGS workflows are highly sensitive, meaning even trace amounts of unwanted DNA can introduce artifacts or noise into the dataset. This can complicate downstream analyses, requiring researchers to implement additional computational filtering to distinguish between genuine genetic signals and contamination. Such corrective measures not only demand advanced bioinformatics expertise but also increase the computational resources and time needed to process the data, driving up costs and potentially delaying critical discoveries or diagnoses.

Prokaryotic DNA contamination in eukaryotic samples (for example, bacterial contamination of human, animal or plant-derived samples), presents unique challenges in NGS workflows. Because bacteria can be so abundant in naturally collected samples, contamination by bacterial DNA can reduce sequencing depth for the target eukaryotic genome, diminishing data quality and forcing labs to perform additional sequencing runs. In clinical applications, bacterial contamination can obscure or mimic human genetic features, leading to the possibility of inaccurate interpretations. These inaccuracies could result in the misidentification of disease-associated mutations or to an increase in the false negative rate, requiring extensive reanalysis or validation efforts to ensure reliable conclusions.

In addition to increasing the financial burden, bacterial contamination reduces the cost-effectiveness of NGS technologies by necessitating stricter quality control measures, such as enhanced sample handling protocols and improved reagent purity. Smaller labs or resource-constrained environments may struggle to implement such measures, further compounding the financial strain and limiting access to NGS technologies. Ultimately, the presence of DNA contamination in sequencing workflows underscores the need for rigorous sample preparation and contamination prevention strategies to safeguard the accuracy and efficiency of genomic studies.

### SUMMARY

In some embodiments, a method is provided for enriching eukaryotic DNA in a DNA sample containing eukaryotic DNA and prokaryotic DNA. A DNA sample comprising eukaryotic DNA and prokaryotic DNA can be accessed. The DNA sample can be treated with one or more restriction enzymes, where at least one of the one or more restriction enzymes is a methylation-specific restriction enzyme that selectively digests prokaryotic DNA by targeting methylation patterns characteristic of prokaryotic genomes, the methylation patterns being absent or rare in eukaryotic DNA. The treated DNA sample can be sequenced.

The methylation-specific restriction enzyme may include DpnI, McrBC, a combination thereof, and/or any other restriction enzyme that targets a methylation pattern unique to prokaryotes.

The methylation pattern targeted by the restriction enzyme may include N6-methyladenine (m6A) or N4-methylcytosine (m4C).

The sequencing may include next-generation sequencing (NGS).

The method may further include separating the digested prokaryotic DNA fragments from the DNA sample prior to sequencing, wherein the separation comprises size exclusion, centrifugation, filtration, or a combination thereof.

The methylation-specific digestion may occur after performing DNA repair and ligation of adaptor sequences and indexes to the DNA fragments.

The DNA sample may be enriched for prokaryotic DNA by treating the DNA sample with one or more restriction enzymes that selectively digest eukaryotic DNA by targeting methylation patterns characteristic of eukaryotic genomes, and/or the digestion may occur after DNA repair and ligation of adaptor and indexing sequences to the DNA fragments.

The DNA sample may be obtained from a saliva sample.

The sequencing may include nanopore sequencing, single-molecule real-time (SMRT) sequencing, or another high-throughput sequencing technology.

The methylation-specific digestion and sequencing steps may be performed sequentially in a single integrated workflow.

The DNA library may be prepared by treating the DNA sample with one or more restriction enzymes and ligating adaptor sequences and indexes, and the prepared library may be stored or transferred to a separate facility for sequencing.

In some embodiments, a kit may be provided for enriching eukaryotic DNA in a DNA sample containing eukaryotic DNA and prokaryotic DNA. The kit may include one or more restriction enzymes, wherein at least one of the restriction enzymes is a methylation-specific restriction enzyme that selectively digests prokaryotic DNA by targeting methylation patterns characteristic of prokaryotic genomes; and instructions for using the kit to enrich eukaryotic DNA by treating the DNA sample with the one or more restriction enzymes prior to sequencing.

The kit may further include reagents for performing DNA repair, adaptor ligation, and indexing of DNA fragments prior to treatment with the one or more restriction enzymes.

The kit may also include buffers, polymerases, or nucleotides optimized for use with the restriction enzymes.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present disclosure is described in conjunction with the appended figures:

FIG. 1 illustrates a comparison of total reads vs. fraction of aligned bases in blood and saliva samples.

FIG. 2 illustrates the different manners in which eukaryotic and prokaryotic genomes are methylated.

FIG. 3 illustrates a strategy for enriching mixed samples for eukaryotic DNA via the use of methylation-specific restriction enzymes.

FIG. 4 illustrates a standard next-generation sequencing library preparation workflow.

FIG. 5 illustrates how methylation-specific restriction enzymes may be incorporated in the library preparation workflow to enrich for eukaryotic DNA.

FIG. 6a shows a list of restriction enzymes that may be used to enrich a sample for eukaryotic DNA.

FIG. 6b shows a continuation of the list of restriction enzymes in FIG. 6a that may be used to enrich a sample for eukaryotic DNA.

FIG. 6c shows a continuation of the list of restriction enzymes in FIG. 6a that may be used to enrich a sample for eukaryotic DNA.

### DETAILED DESCRIPTION

Human DNA samples can often be contaminated with bacterial (prokaryotic) DNA, presenting challenges in situations where the goal is to sequence the human genome or analyze human-specific genetic material. For example, infected tissue samples, such as biopsies from abscesses or wounds, frequently contain bacterial DNA due to the presence of pathogens. In such cases, if the aim is to study the host's genetic or epigenetic response to infection, bacterial DNA can obscure the human data and must be removed. Similarly, blood samples from patients with sepsis or bacteremia contain bacterial DNA from circulating pathogens, which can interfere with analyses focused on human cell-free DNA, such as those used for cancer diagnostics or prenatal testing.

In environmental or forensic contexts, human DNA is often isolated from highly contaminated samples, such as soil-covered remains or ancient DNA preserved in archaeological sites. These samples typically contain significant bacterial DNA introduced from the surrounding environment or through microbial activity during decomposition. If the primary objective is to recover and sequence human DNA for genetic or anthropological studies, the bacterial DNA must be excluded to ensure accurate results. Similarly, postmortem samples frequently harbor bacterial DNA due to microbial proliferation during decomposition, which can hinder efforts to extract human genetic material for forensic investigations.

Saliva samples, commonly used for genetic testing or ancestry analysis, also present a challenge when bacterial DNA dominates the sample. While human DNA in saliva is derived from epithelial cells, bacterial DNA from the oral microbiome often constitutes a significant proportion of the total DNA. When the focus is on sequencing human DNA, the bacterial component must be carefully removed to ensure accurate results. In all these cases, bacterial DNA contamination reduces the effective concentration of human DNA and can compromise the success of sequencing efforts if not properly addressed.

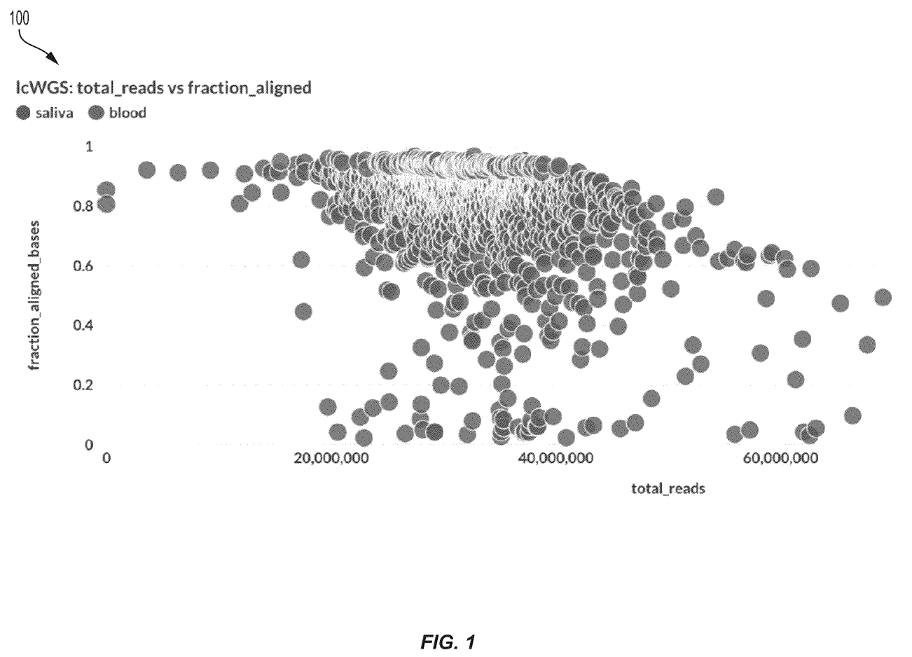

FIG. 1 illustrates a comparison between a fraction of aligned bases (y-axis) and a total number of sequencing reads (x-axis) for saliva and blood samples. Each data point represents an individual sample, with red points indicating blood and blue points indicating saliva. Blood samples exhibit consistently high alignment fractions, clustering close to 1, regardless of the total number of reads, suggesting a high proportion of human DNA in these samples. In contrast, saliva samples display a much wider range of alignment fractions, with many falling significantly below 1 and some nearing 0. This suggests that a substantial portion of the sequencing reads in saliva samples do not align to the human reference genome, likely due to bacterial contamination from the oral microbiome. Even saliva samples with a high number of total reads show poor alignment in some cases, reinforcing the idea that bacterial DNA is reducing the effective concentration of human DNA available for analysis.

Post-processing data analysis may be used to exclude reads that fail to align to the human genome, ensuring that downstream analyses focus only on high-quality human sequences. This process, however, is computationally expensive and time-consuming, requiring significant resource allocation. Filtering out bacterial reads involves extensive computational steps, such as mapping reads to a reference genome and discarding non-human sequences, which can dramatically increase processing time. Additionally, the need for extra computational resources and storage to handle the raw and filtered data adds to the overall cost.

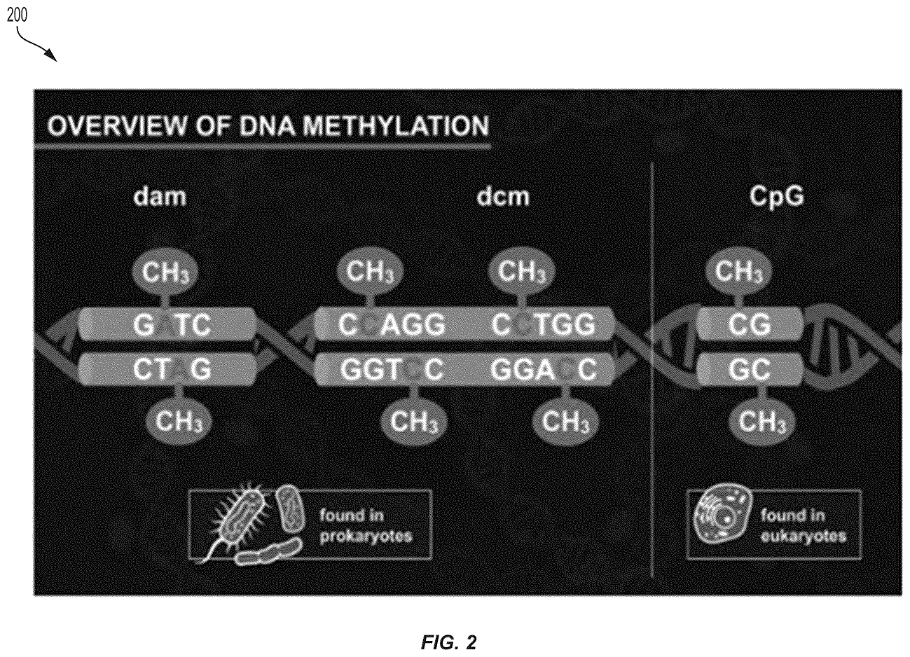

In some embodiments, the invention employs methylation-specific restriction enzymes to enrich the target DNA in a NGS sample by degrading bacterial or prokaryotic DNA. In this example, the target DNA may represent human DNA or DNA from any other eukaryotic species. Methylation-specific restriction enzymes are specialized proteins that recognize and cut DNA at specific sequences, depending on whether those sequences are methylated or unmethylated. FIG. 2 shows the different manners in which eukaryotic and prokaryotic DNA is methylated. Prokaryotic and eukaryotic DNA exhibit distinct methylation patterns, which influence how they interact with methylation-specific restriction enzymes. In prokaryotic DNA, methylation often occurs on adenine bases within sequences like GATC, mediated by enzymes such as Dam (DNA adenine methyltransferase). Some methylation-specific restriction enzymes can recognize and cleave methylated sites, enabling targeted DNA cleavage in a phylogenetically specific manner. Examples of such enzymes are Dpnl and McrBC. In contrast, eukaryotic DNA methylation typically occurs on cytosine bases within CpG dinucleotides. These differences in methylation patterns and enzymatic recognition underline the distinct genomic features of prokaryotic and eukaryotic organisms.

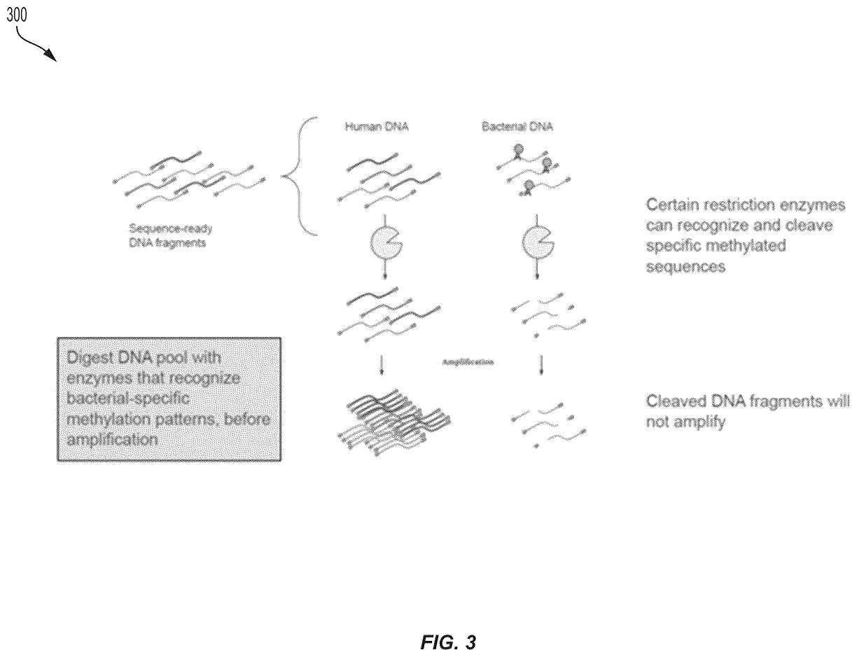

FIG. 3 shows a diagram of one embodiment of the invention in which methylation-specific restriction enzymes are used to enrich a sample of sequence-ready DNA fragments. The DNA fragments in this example contain a mixture of target human DNA of interest and bacterial DNA as a contaminant. In FIG. 3, the DNA fragments have undergone repair and been ligated to the appropriate adaptors (gray boxes at the end of the DNA fragments) and indexes for NGS. Adaptor sequences are short, synthetic DNA or RNA oligonucleotides ligated to the ends of DNA or RNA fragments to enable amplification, sequencing, and identification during next-generation sequencing workflows. The bacterial DNA is shown as methylated (circles attached to the DNA) while the human DNA is not. The methylation shown here is specific to the bacterial methylation sites and is recognized specifically by bacterial restriction enzymes. The human DNA in an actual sample is likely also methylated, but specifically at CpG sites that are not recognized by these same restriction enzymes. Bacterial methylation-specific restriction enzymes are then added to the mixed sample and only the bacterial DNA cleaved. The human or eukaryotic sample may then be amplified and sequenced.

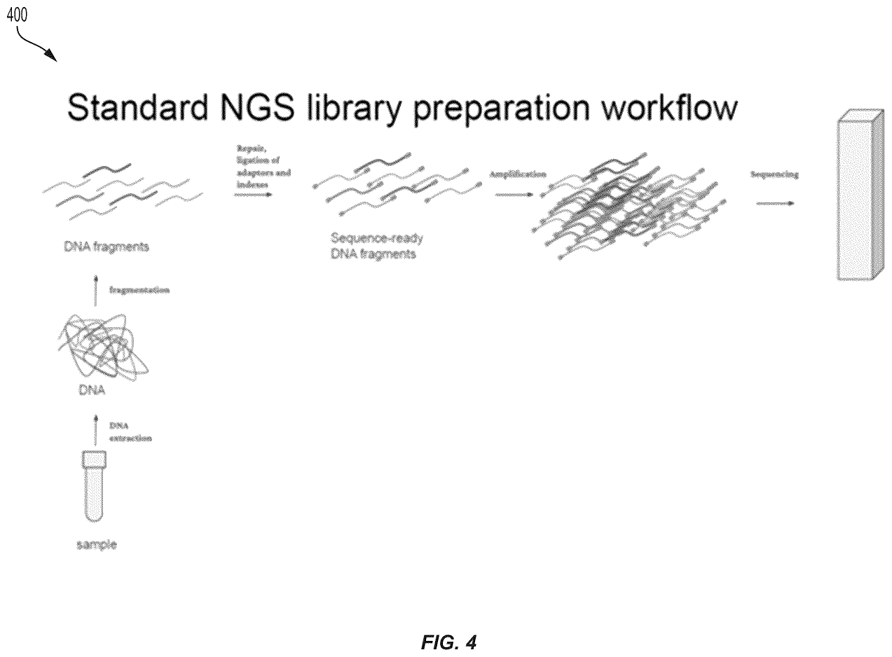

FIG. 4 illustrates a high-level summary of the workflow involved in DNA sequencing, particularly next-generation sequencing (NGS). A DNA sample is first fragmented using a suitable enzyme, such as but not limited to, DNase I, Fragmentase, and Tn5 transposase, or with any other fragmentation technique such as sonication. The DNA sample may then undergo end repair to ensure the DNA fragments have uniform, blunt ends by utilizing polymerase and exonuclease activities to fill in overhangs or trim excess bases. This process prepares the fragments for downstream adaptor ligation by creating compatibility between the DNA ends and the adaptors. A-tailing follows end repair and involves the addition of a single adenine (A) nucleotide to the 3′ ends of the DNA fragments. This step prevents fragments from self-ligating and enables the ligation of adaptors with complementary thymine (T) overhangs, a common feature of library construction. Preparation of the DNA sample often includes DNA damage repair, employing enzymatic treatments to correct single-strand nicks, oxidized bases, or other chemical modifications that can compromise the integrity of DNA. For multiplexed sequencing workflows, index sequences are often included in the adaptors. These short, unique sequences enable the identification and separation of multiple samples that are pooled together during sequencing. Together, these processes improve the quality and versatility of the DNA library for accurate sequencing.

FIG. 4 further illustrates the amplification step in the processing of a DNA sample prior to sequencing. In NGS, amplification typically occurs on a flow cell via a process called bridge amplification. Bridge amplification generates clusters of identical DNA fragments on a solid surface, such as a flow cell. During this process, the adaptor-ligated DNA fragments hybridize to complementary oligonucleotides immobilized on the surface, and a polymerase synthesizes a complementary strand. The resulting double-stranded DNA denatures, and the single strands form “bridges” by bending to hybridize with nearby oligonucleotides. Repeated cycles of amplification produce dense clusters of DNA copies, which are crucial for generating a strong and measurable sequencing signal. Upon completion of the amplification process, the amplified DNA samples may be subsequently sequenced using any NGS platform, such as the Illumina Next Generation Sequencing Platform®.

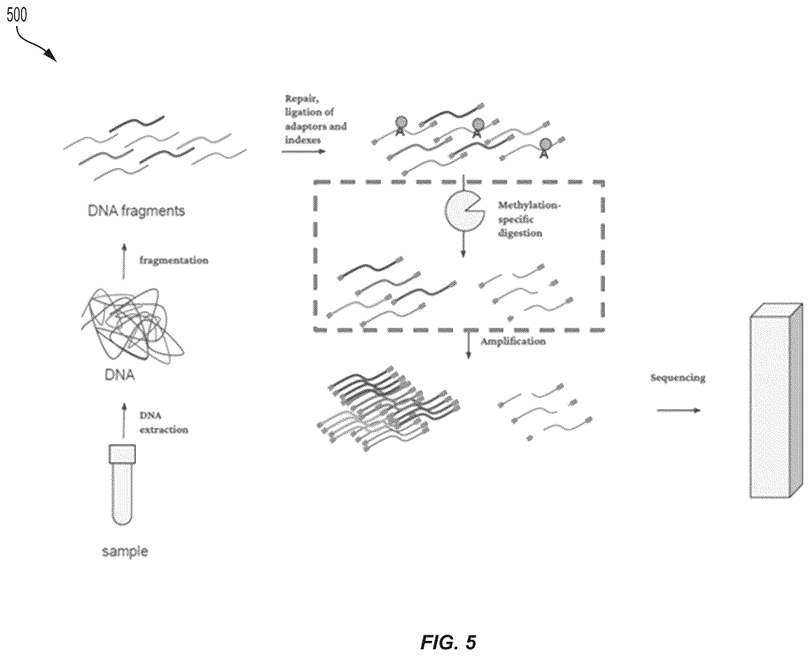

FIG. 5 shows that, in some embodiments of the invention, an additional preprocessing step is performed during the preparation of the DNA library prior to amplification and sequencing. If the DNA sample is heterogenous and contains target DNA fragments from one or more patients (or any eukaryotic subject(s)) and contaminating prokaryotic DNA, an extra DNA digestion step may be used to enrich the sample for the eukaryotic component. In some embodiments, this is achieved after the process of DNA repair and adaptor and index ligation. The process of DNA repair helps stabilize the fragments and prevents degradation of the sample. Digesting the sample using one or more methylation-specific restriction enzymes after the DNA repair process, as opposed to prior, provides the technical advantage of stabilizing the DNA fragments preventing further sample degradation of the target eukaryotic DNA during the digestion of the contaminant prokaryotic DNA.

FIG. 5 further illustrates that DNA fragments degraded via methylation-specific restriction enzymes will not undergo significant amplification with a typical NGS flow cell protocol. Degraded fragments will not amplify despite having undergone adaptor sequence ligation because proper clonal amplification through bridge amplification on a flow cell requires adaptor sequences on both the 3′ and 5′ ends of the DNA fragment. While the contaminant prokaryotic DNA may occupy oligonucleotide anchors (or probes) on the flow cell and compete with target eukaryotic DNA, the DNA library as a whole generally anneals to a very small faction of the flow cell's available anchors (approximately 1-5%), with most anchoring sites ultimately occupied by fragments resulting from the amplification process.

It will be appreciated that in some embodiments other preprocessing steps may be performed in combination with the methods described herein to further enrich the sample for target eukaryotic DNA in the final DNA library prior to sequencing. For instance, it will be appreciated that standard techniques such as sorting the DNA fragments by size after digestion of contaminant DNA by methylation-specific restriction enzymes may be used to remove the prokaryotic DNA fragments. An exemplary, but not inclusive, list of such preprocessing techniques that may be combined with the techniques described herein include differential lysis to selectively break open prokaryotic cells, enzymatic digestion of extracellular DNA to degrade bacterial DNA, centrifugation or filtration to separate human and prokaryotic components, magnetic capture of methylated DNA, and the use of human-specific DNA capture probes to enrich for human DNA while depleting microbial sequences.

It will be appreciated that the methods and techniques of the invention described within may be applied to any sequencing strategy, NGS or otherwise, that relies on the ligation of adaptor sequences on both ends of the DNA fragments to achieve amplification before sequencing. In addition to the Illumina Sequencing Platform, such techniques and platforms may include Ion Torrent (Thermo Fisher), Roche 454, Pacific Biosciences (PacBio), Oxford Nanopore Technologies, and BGI/MGI sequencing platforms. As digestion with the one or more methylation-specific restriction enzyme results in smaller fragments without adaptor sequences at both the 3′ and 5′ ends, amplification will not occur, and the sequencing results will be enriched for the eukaryotic target sequence.

The methods described herein may also be applied to sequencing strategies that either require ligation of adaptors at only one end of the DNA fragment or require no ligation of traditional adaptor sequences. For example, Oxford Nanopore Technologies can sequence DNA fragments with a single adaptor attached, as seen in certain rapid sequencing protocols where one end remains free. Some single-primer extension (SPE) methods also involve adaptor ligation to only one end of the DNA fragment, enabling linear amplification rather than the typical double-ended amplification approach. These alternative strategies are tailored for specific applications and offer simplified workflows, though they may have trade-offs in efficiency or accuracy compared to systems that use dual adaptors. It will be appreciated that some embodiments of the invention described herein may also serve to enrich DNA libraries for eukaryotic fragments when using these or similar sequencing strategies. For instance, digestion of the prokaryotic DNA fragments can still enrich the library for the target DNA if the digested fragments can be removed after digestion using a number of strategies, such as selecting by size.

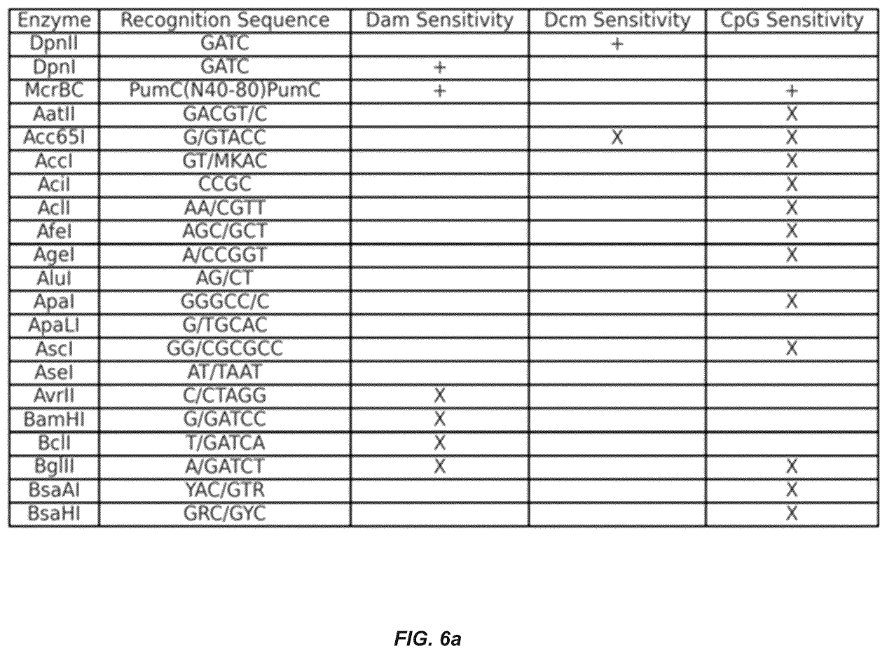

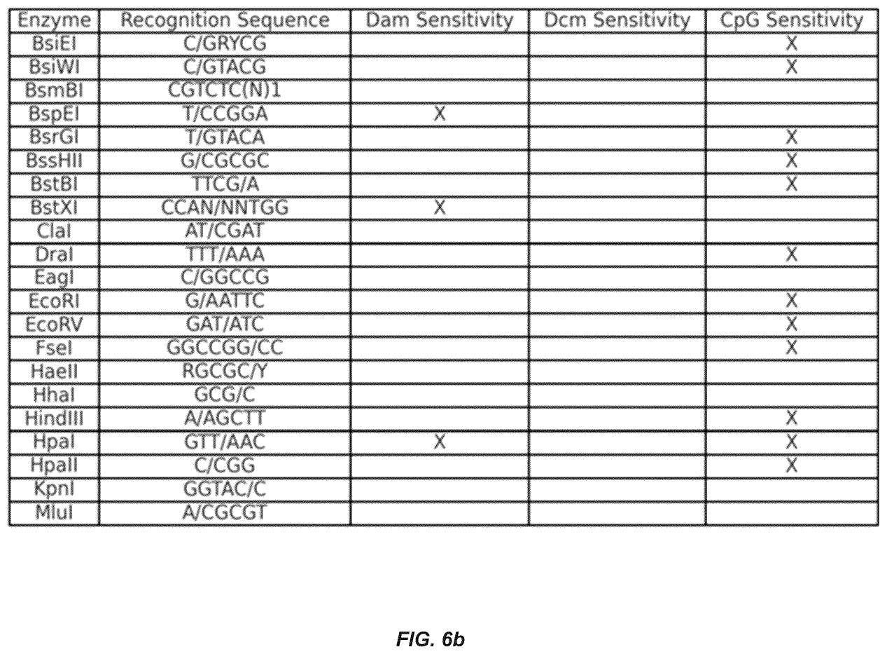

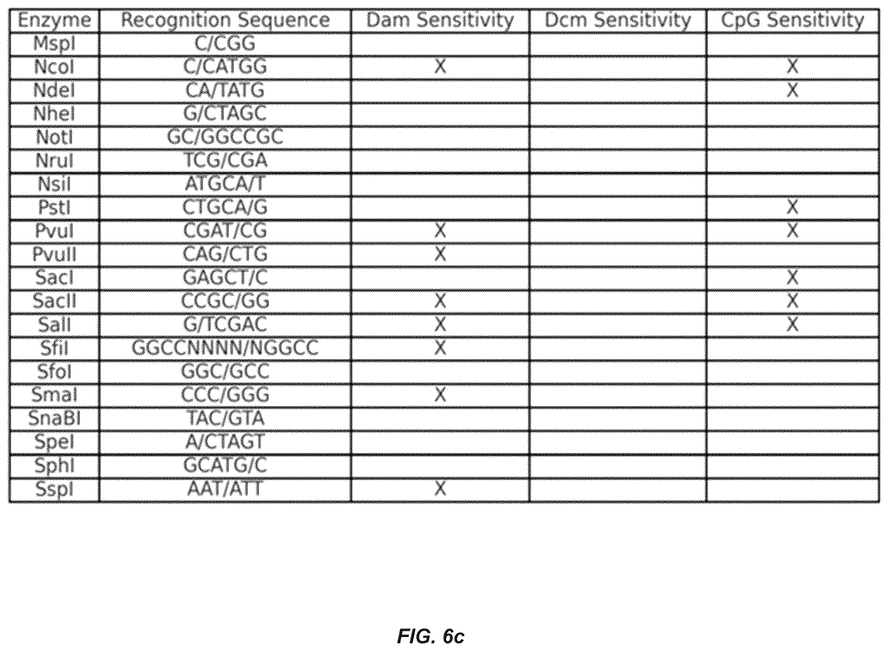

FIGS. 6a-c show an exemplary list of prokaryotic-specific restriction enzymes that may be suitable for the eukaryotic enrichment step described in some embodiments of the invention. It will be appreciated that these enzymes may work in a methylation-specific manner or not, but that any enzyme capable of acting in a phylogenetic specific manner may be suitable for the enrichment step described herein. In FIGS. 6 a-c, an X represents a suppression of digestion by the restriction enzyme if the DNA is methylated at that site (Dam, Dcm, or CpG). A blank cell illustrates no sensitivity to methylation for that restriction enzyme at that site. A + represents an enhancement of digestion by the restriction enzyme if the site is methylated. Note that the Dam and Dcm recognition sites are specific to prokaryotic genomes, and CpG is eukaryotic-specific. The list is not comprehensive for prokaryotic-specific restriction enzymes regardless of their dependence on methylation states. It will be appreciated that for the purpose of enriching the DNA library, a single restriction enzyme or any combination of restriction enzymes, regardless of dependency on methylation states, may be utilized.

It will be appreciated that the methods and techniques of the invention described within could be performed to enrich prokaryotic DNA in the sample by utilizing a methylation-specific restriction enzyme to digest eukaryotic DNA at GpC sites or other eukaryotic specific methylation sites. In such cases, library preparation may proceed as described previously here or elsewhere. The digested eukaryotic fragments may be removed via a variety of methods, including selection for size, or they may remain in the sample but fail to achieve standard amplification or bridge amplification as described above.

The methods described herein provide a technical advantage over existing methods for reducing the influence of contaminating prokaryotic DNA in a heterogeneous DNA sample. The inventions described herein remove contaminant DNA prior to sequencing and ensure that sequencing resources are focused on the target DNA, improving efficiency and cost-effectiveness. Contaminant DNA consumes valuable sequencing capacity, reducing the number of reads available for the DNA of interest and potentially leading to insufficient depth for comprehensive analysis. Computational methods for removing contaminants post-sequencing rely on reference databases, which may be incomplete or inaccurate, increasing the risk of misclassification or incomplete removal of contaminant sequences. By removing contaminant DNA before sequencing, the resulting dataset is more accurate, efficient, and representative, ensuring reliable outcomes for a wide range of applications, including research, clinical diagnostics, and medical analyses.

It will be appreciated that in some embodiments the sample will include a saliva sample. In this exemplary case, the sample will likely harbor bacterial DNA from the oral microbiome as a significant contaminant. DNA sequencing on saliva samples is popular because it is a non-invasive, convenient, and cost-effective method for collecting DNA. Unlike blood draws, which require trained professionals and sterile environments, saliva collection can be done easily at home using collection kits. Saliva contains sufficient DNA from both human epithelial cells and microbial cells, making it suitable for various applications, including ancestry testing, disease risk profiling, and microbiome analysis. Additionally, saliva is stable when preserved with the right storage solutions, enabling reliable DNA extraction even after shipping and storage. This accessibility and ease of use make saliva an ideal choice for large-scale genomic studies and consumer genetic testing services.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed:

1. A method for enriching eukaryotic DNA in a DNA sample containing eukaryotic DNA and prokaryotic DNA, comprising:
accessing a DNA sample comprising eukaryotic DNA and prokaryotic DNA;
treating the DNA sample with one or more restriction enzymes, wherein at least one of the one or more restriction enzymes includes a methylation-specific restriction enzyme that selectively digests prokaryotic DNA by targeting methylation patterns characteristic of prokaryotic genomes, the methylation patterns being absent or rare in eukaryotic DNA; and
sequencing the treated DNA sample.

2. The method of claim 1, wherein the methylation-specific restriction enzyme comprises DpnI.

3. The method of claim 1, wherein the methylation-specific restriction enzyme comprises McrBC.

4. The method of claim 1, wherein the methylation pattern targeted by the restriction enzyme comprises N6-methyladenine (m6A).

5. The method of claim 1, wherein the methylation pattern targeted by the restriction enzyme comprises N4-methylcytosine (m4C).

6. The method of claim 1, wherein the sequencing comprises next-generation sequencing (NGS).

7. The method of claim 1, further comprising separating the digested prokaryotic DNA fragments from the DNA sample prior to sequencing, wherein the separation comprises size exclusion, centrifugation, filtration, or a combination thereof.

8. The method of claim 1, wherein the methylation-specific digestion occurs after performing DNA repair and ligation of adaptor sequences and indexes to the DNA fragments.

9. The method of claim 1, wherein the DNA sample is enriched for prokaryotic DNA by treating the DNA sample with one or more restriction enzymes that selectively digest eukaryotic DNA by targeting methylation patterns characteristic of eukaryotic genomes, and wherein the digestion occurs after DNA repair and ligation of adaptor and indexing sequences to the DNA fragments.

10. The method of claim 1, wherein the DNA sample is obtained from a saliva sample.

11. The method of claim 1, wherein the sequencing comprises nanopore sequencing.

12. The method of claim 1, wherein the sequencing comprises single-molecule real-time (SMRT) sequencing.

13. The method of claim 1, wherein the methylation-specific digestion and sequencing are performed sequentially in a single integrated workflow.

14. The method of claim 1, wherein the DNA library is prepared by treating the DNA sample with one or more restriction enzymes and ligating adaptor sequences and indexes, and the prepared library is stored or transferred to a separate facility for sequencing.

15. A kit for enriching eukaryotic DNA in a DNA sample containing eukaryotic DNA and prokaryotic DNA, comprising:
one or more restriction enzymes, wherein at least one of the restriction enzymes is a methylation-specific restriction enzyme that selectively digests prokaryotic DNA by targeting methylation patterns characteristic of prokaryotic genomes; and
instructions for using the kit to enrich eukaryotic DNA by treating the DNA sample with the one or more restriction enzymes prior to sequencing.

16. The kit of claim 15, further comprising one or more reagents for performing DNA repair, adaptor ligation, and indexing of DNA fragments prior to treatment with the one or more restriction enzymes.

17. The kit of claim 15, further comprising a buffer optimized for use with the restriction enzymes.

18. The kit of claim 15, further comprising a polymerases for use with the restriction enzymes.

19. The kit of claim 15, further comprising a nucleotide optimized for use with the restriction enzymes.
